(19) United States
(12) Patent Application Publication
(10) Pub. No.: US 2003/0124087 A1
(43) Pub. Date: Jul. 3, 2003

(54) Anti-adhesion barrier

(75) Inventors: Dae Sik Kim, Jeonju (KR); Do Hoon Kim, Gwangju (KR); Jeong Kwon Lee, Nonsan (KR)

Correspondence Address: STAAS & HALSEY LLP, 700 11TH STREET, NW, SUITE 500, WASHINGTON, DC, 20001, US

(73) Assignee: Amitie Co. Ltd., Dukjin-gu (KR)

(21) Appl. No.: 10/316,853

(22) Filed: Dec. 12, 2002

(30) Foreign Application Priority Data
Dec. 26, 2001 (KR) 2001-85142
Oct. 28, 2002 (KR) 2002-65986

(51) Int. Cl. A61K 31/74; A61K 31/727; A61K 31/717; A61K 38/20; A61K 31/56; A61K 31/198

(52) U.S. Cl. 424/78.18; 514/54; 514/56; 514/57; 424/85.2; 514/355; 514/59; 514/171; 514/565; 424/718

(57) ABSTRACT

The present invention relates to anti-adhesion barriers which prevents an adhesion between tissues at an operated legion and have various shapes, comprising sodium carboxymethyl cellulose (NaCMC) and gellan gum in 1:(0.1˜10) of weight ratio.

The anti-adhesion barriers of the present invention prevent adhesions at the operated legion or between bleeding legion and existed tissues more effectively after self-transplantation and being sealed completely, than conventional anti-adhesion barriers do and moreover, have remarkable haemostatic effects, anti-inflammation adhesion and application effect.

### FIELD OF THE INVENTION

The present invention relates to anti-adhesion barriers which suppress bleeding at an operated legion (haemostatic effect) and prevent a mutual adhesion of tissues at the operated legion (anti-adhesion effect) remarkably.

### BACKGROUND

Generally, if there is a slight bleeding after or during an operation, a number of problems are caused in re-operations. The operated legion and the bleeding legion are adhered with cells existed in tissues even after completed to be sutured.

In practice, the adhesions are reported to be provoked in 70˜95% of frequency after abdominal operations (Eur. J. Surg., 1997, Suppl., 577, 10-16, 24-31, 32-39). Especially, in the abdominal operation the mutual adhesion of organs such as adhesion between abdominal cavity wall and intestine is caused and thus more disadvantageous.

In order to solve these problems, many researches have been tried actively to develop anti-adhesion barriers which can be degraded in a body and have a little toxicity and to prevent mutual adhesions of organs for a short time or a long time.

For purpose of an anti-adhesion barrier, several characteristics such as good adhesiveness, applicability, affinity to tissues, anti-bacterial property and the like as well as capacities for suppressing a mutual adhesion of organs and preventing a bleeding are required.

Recently, various studies have been attempted a lot to develop anti-adhesion barriers which can block a contact onto a legion site after an operation. In this anti-adhesion barrier, bioadsorptive polymer having a carboxyl terminal group in a high molecular weight is utilized and can be hydrated within a body. Then, the legion site is separated from adjacent tissues during a treatment period for the legion so that the caused adhesion is prevented.

Moreover, after the treatment is completed, the anti-adhesion barrier might be absorbed and removed naturally, which does not affect onto normal tissues.

U.S. Pat. No. 4,141,973 has disclosed that biodegradable polymer such as hyaluronic acid (HA) could be used for this method. Unfortunately, HA cannot be applied to an anti-adhesion barrier in practice since it may be degraded and absorbed into a body at a relatively high speed.

In the meantime, sodium carboxymethyl cellulose (NaCMC) has been reported as a substance for an anti-adhesion agent in various cases (Fertil. Steril., 1984, June, Gynecol., 1986, 155: 3, 667-670). However, it is known that sodium carboxymethyl cellulose is difficult to be applied to an anti-adhesion barrier since it is absorbed or removed too fast into a body and thus cannot have a preventive effect upon the adhesion.

As demonstrated above, in order to solve problems of prior arts, there are many trials for effects upon adhesion which is difficult to be manifested due to excessive rapidity of degradation or removal in material of anti-adhesion barriers. Concretely, the method for forming a connection between biodegradable polymers has been illustrated. Precisely, EP 507,604 has demonstrated that polysaccharides having a carboxyl terminus were bonded with polyvalent metal ions, which reduces the solubility so as to be utilized as an anti-adhesive barrier. In addition, U.S. Pat. No. 5,318,780 has illustrated that the anti-adhesive barrier was prepared by using in situ gelatin method.

However, these methods are concluded not to be suitable for an anti-adhesive barrier since they reduce tissue adhesive strength and complicate the process for an operation although they are advantageous to make films within a body by using membrane forming polymer (hydroxypropyl methyl cellulose, HPMC).

On the other hand, U.S. Pat. Nos. 5,017,229; 5,527,893; 5,760,200 and so on have disclosed that a kind of polysaccharide such as hyaluronic acid (HA) and carboxymethyl cellulose (CMC) should be utilized to prevent an adhesion. These methods also have some problems in the procedure for removing biologically toxic material generated in the process for the preparation. Hydration is proceeded to make gelatin induced at a high speed, which causes some difficulties in the treatment and the operation, namely preventing a reoperation after separating them again (Surg. Clin. Nor. Am., 1997, 77: 3, 671-688).

General adhesion of hydrophilic biopolymer against body tissues could be affected most by hydration but polymers having a carboxyl terminus bonding with tissues are suitable for this use. Moreover, in case of gel type the adhesiveness of polymer itself is more important factor since already saturated with moisture.

Recently, several operational techniques have been developed and advantages preventing adhesion are acquired additionally (Hepato-Gastroenterol., 1991, 38, 283). This trend for developing anti-adhesion barriers meet changes of these operational techniques and continues to develop anti-adhesion barriers in a solution or a gel type, although solution type, gel type and film type are known to types of anti-adhesion barrier (The Adhesion Prevention Opportunity Report from MDI, 1998).

The gel type anti-adhesion barrier attracts attention recently since it can prevent an adhesion effectively with treating legion sites in a small amount. Especially, pharmaceutics which can be applied in a gel type restrictively to spine operation, tendon operation and the like have been attempted (U.S. Pat. No. 5,605,938). Presently, commercially available phosphated saline manufactured in Gliatech company and a gel type anti-adhesion barrier consisting in porcine and polyglycan ester (trademark: ADCON-L) have been utilized widely.

Unfortunately, typical anti-adhesion barriers described above are still restricted in their efficacies such as adhesiveness, affinity, anti-inflammation, anti-bacteriai property and regeneration in spite of achievements in anti-adhesiveness and haemostatic function.

### SUMMARY OF THE INVENTION

In order to overcome disadvantages of conventional anti-adhesion barriers described above, the present invention provides an effective anti-adhesion barrier having several features such as anti-adhesion efficacy, haemostatic property, affinity and so on, anti-inflammation, anti-bacterial property and regenerative function at a legion site.

Precisely, the object of the present invention is to provide new anti-adhesion barrier in which efficacies of sodium carboxymethyl cellulose (NaCMC) known to have anti-inflammation, anti-bacterial property, regenerative property, haemostatic property as well as remarkable suitability and safety should be sustained and several limitations of anti-adhesion barrier such as degradation and absorption be satisfied so as to become safe and effective.

### BRIEF DESCRIPTION OF THE DRAWINGS

The above and other objects, features and other advantages of the present invention will be more clearly understood from the following detailed description taken in conjunction with the accompanying drawings, in which;

FIG. 1 depicts the experimental result that the anti-adhesion barrier of the present invention can suppress a cell growth.

FIG. 2 depicts the effect upon anti-adhesion when the anti-adhesion barrier of the present invention is applied to the operation of panniculus adipose.

FIG. 3 depicts the effect upon anti-adhesion when the anti-adhesion barrier of the present invention is applied to the operation of spine muscles.

FIG. 4 depicts the effect upon anti-adhesion when the anti-adhesion barrier of the present invention is applied to the operation of abdominal cavity.

FIG. 5 depicts the effect upon anti-adhesion when the anti-adhesion barrier of the present invention is applied to the operation of dorsal muscles.

FIGS. 6a and 6b depict the absorption states of anti-adhesion barrier in a body according to a time lapse in the present invention.

### DETAILED DESCRIPTION OF PREFERRED EMBODIMENTS

In order to attain the above—mentioned objects of the present invention, the present invention provides an anti-adhesion barrier comprising (1) sodium carboxymethyl cellulose (NaCMC) which has a regenerative function in a legion site, haemostatic property, compatibility for a human body, and safety and (2) gellan gum which controls an absorption of sodium carboxymethyl cellulose into a human body, sustains a gel shape formed in a body and enhances a lubrication, in a weight ratio of 1 (0.2˜5). At this moment, the composition of NaCMC and gellan gum is preferable to be in between 1:(0.8˜1.25) and namely, the ratios are almost the some.

Precisely. sodium carboxymethyl cellulose (hereinafter, referred to as “NaCMC”) plays a role as a matrix to form gel having an effect for preventing an adhesion in the present invention. Gellan gum plays a role to situate body tissues onto a right position by conferring a lubricous property on gel as well as to sustain a gel shape during a required period by preventing a rapid degradation and absorption of NaCMC.

In the components applied to the composition of the present invention, the molecular weight is not considered as an important factor conventionally, but preferably in case of NaCMC, the molecular weight is about 100,000˜1,000,000 Da and in case of gellan gum, about 200,000˜1,000,000 Da. Besides, the molecular weight and the composition of each component can be controlled properly, depending upon a legion site and a purpose of use. For example, while administered onto sites having many trauma and bleeding, the molecular weight and the composition of NaCMC is preferable to become higher relatively and while administered onto internal organs, the molecular weight and the composition of NaCMC is preferable to be lower relatively.

The condition that the molecular weight or the composition of NaCMC is too high is not preferable, which is likely to block the recover of organs after the operation. When the molecular weight or the composition of gellan gum is too high is too high, tissues can be also interrupted to be returned to a previous position after an operation and the operated sites can be rather inhibited to be recovered, since the formed gel is sustained for a longer period than a needed period. On the other hand, if the molecular weight or the composition of components in each anti-adhesion barrier, the efficacy of the anti-adhesion barrier become non-effective in its functions.

Especially, if the molecular weight or the composition of NaCMC is toc low, some problems that the prevention of adhesion, haemostatic efficacies and the like would be decreased can be caused.

Concretely, the gel type of anti-adhesion barrier illustrated below, can be utilized after the operation of peritoneal cavity organs. At this moment, the concentrations (compositions) of NaCMC and gellan gum are preferable to be adjusted to 5.0 weight % respectively (by using distilled water or small amount of additives), in which the anti-adhesion barrier play a role to lubricate and then to improve the return to original position. The adequate viscosity of the anti-adhesion barrier confers the effectiveness for preventing an adhesion after an operation.

In the meantime, in order to enhance the efficacy of anti-adhesion, the anti-adhesion barrier of the present invention is composed of additionally of various kinds of natural substances which are known to nontoxic to a human body.

Precisely, one or more than two substances selected from a group comprising interleukine-10, hyaluronate, heparin, nifedipine HCl, phosphatidylcholine, medroxyprogestone acetate, L-arginine, nitric oxide, polyvinylalcohol (PVA), protein inhibitor and dextran are added to the anti-adhesion barrier of the present invention in a weight ratio of 1:(0.2˜5) against sodium carboxymethyl cellulose. At this moment, the protein inhibitor can be selected among tumor necrosis factor (TNF), INF, IL-10, IL-13, IL-1, interferone and the like.

Furthermore, in order to sterilize microbes infected in the process of operation, the anti-adhesion barrier of the present invention can contain antibiotics additionally in a weight ratio of 1:(0.02˜0.06) against sodium carboxymethyl cellulose.

At this moment, the antibiotics can be adopted among penicillins, cephalosporins, aminoglycosides, tetracyclins, chloroamphenicols, quinines, polypeptide system antibiotics and the like. These antibiotics can be added in adequate compositions and processes according to respective characteristics and activities.

On the other hand, the anti-adhesion barrier prepared in the present invention can have various forms and preferably, a foam type, powder type, gel type or the like.

The foam type of anti-adhesion barrier is prepared by a process, comprising steps as follows: (1) making a mixed solution consisting in the above mentioned components and an adequate amount of water; (2) injecting the mixed solution into a container having a suitable shape and size; (3) freezing rapidly by submersing into liquid nitrogen; and (4) lyophilizing under a reduced pressure at less than −55° C. for more than 48 hours. As a result, the foam type of anti-adhesion barrier can be prepared to have various shapes and sizes, in accordance with the shape and size of the container. Depending upon requirements, the mixed solution can be more sterilized at the temperature range of 120˜150° C. for 5˜20 minutes by autoclaving, before the step (3) freezing rapidly.

The powder type of anti-adhesion barrier is prepared by pulverizing the foam type of the anti-adhesion barrier manufactured above so that the radius of particles reach 0.2˜0.6 mm. The gel type of anti-adhesion barrier is prepared by the process comprising: (1) adding sterilized water in (4˜19) of weight portion the powder type of anti-adhesion barrier manufactured above; and (2) stirring.

In order to prevent the contamination by microbes, the above anti-adhesion barrier is preferably prepared under a sterilized condition. In addition, in case of a foam type and a powder type, the anti-adhesion barrier is more sterilized with oxidized ethylene gas (EtO gas) and preferably, when distilled water and the powder type anti-adhesion barrier are mixed in case of a gel type, adequate kinds and compositions of antibiotics are added. Depending upon cases, before lyophilizing during the process for preparing the powder type anti-adhesion barrier, adequate kinds and compositions of antibiotics can be added into the autoclaved mixed solution.

Each type anti-adhesion barrier of the present invention play the same role, except that the gel type can be applied onto an operation site in a gelatinous state and the foam type and the powder type become gelatinous by absorbing body fluid and secreted blood components after applied to a operated legion. Therefore, it can be applied properly for the operation, considering the morphological characteristics.

That is to say, the gel type anti-adhesion barrier can be administered by covering all the operated legions as long as it is not flowed downward. The foam type anti-adhesion barrier is convenient to be applied when the space formed at an operated legion is defined accurately. For example, it can be utilized in spine or bone operations. Namely, in case that the operated portion is a crevice formed between bones, it can be applied by being modified to a proper shape and inserted. The powder type anti-adhesion barrier is utilized by scattering at operated legions such as panniculus adiposus, uterus, heart, organs and the like or at portions that the gel is flowed downward.

The anti-adhesion barrier of the present invention has been confirmed to have a preventive effect upon an adhesion between cells in in vitro experiments and not to affect cells negatively (toxicity). In addition, the anti-adhesion barrier of the present invention is remarkable in anti-inflammation, anti-bacterial property, a regenerative function at a legion site, haemostatic property, has an outstanding compatibility for a human body, and a high lubricous property, adhesion property, affinity and the like and also has a good anti-adhesion between cell tissues and haemostatic effects.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

In the following Application Examples, the anti-adhesion barrier having a particular shape is utilized but is natural to be modified into various shapes, depending upon requirements, especially for those skilled in this arts.

### Preparation Example 1

### Preparation of Anti-Adhesion Barrier

The anti-adhesion barriers were prepared according to compositions indicated in the following Preparation Example of standard group and Preparation Example 1˜14. At this moment, NaCMC and gellan gum were adopted to have the average molecular weight of 250,000 Da and 500,000 Da respectively.

TABLE 1

| | NaCMC | gellan | |
|---|---|---|---|
| Examples | (g) | gum (g) | Others (g) |
| Preparation | 10.0 | — | — |
| Example of | | | |
| standard group | | | |
| Preparation | 5.0 | 5.0 | — |
| Example 1 | | | |
| Preparation | 10.0 | 5.0 | — |
| Example 2 | | | |
| Preparation | 15.0 | 5.0 | — |
| Example 3 | | | |
| Preparation | 5.0 | 5.0 | interlukin-10, |
| Example 4˜14 | | | hyaluronate, heparin, |
| | | | nifedipine HCI, |
| | | | phosphatidylcholine, |
| | | | medrexyprogestone acetate, |
| | | | L-arginine, nitric oxide, |
| | | | PVA, protein inhibitor, |
| | | | dextran 5.0 |

Finally, the manufactured shapes were made to be a powder described below in (1) or a foam described in (2).

(1) each component was dissolved in 90 ml of water, freezed rapidly by submersing the solution into liquid nitrogen and then lyophilized at less than −55° C. for more than 48 hours. Afterward, the solid matter was produced and pulverized to make a powder for anti-adhesion barrier.

(2) each component was dissolved in 90 ml of water, injected into a mold with a proper shape, freezed rapidly by submersing the solution into a liquid nitrogen and then lyophilized at less than −55° C. for more than 48 hours. Afterward, the foam for an anti-adhesion barrier was produced. Naturally, it can be pulverized to make a powder like an anti-adhesion barrier of (1).

Depending upon cases, in the above mentioned processes (1) and (2), the sterilization step can be performed at 120° C. for 10 minutes before lyophilizing the solution.

### Application Example

### In vitro Experiments

The anti-adhesion effects were examined by using products manufactured in the above Preparation Examples in a experimental level. Precisely, the experiments for preventing a diffusion by cell growth were performed.

Above all, the powder for an anti-adhesion barrier manufactures in the above mentioned Preparation Example were dissolved with proper amounts of water as demonstrated in Table 2 so as to prepare gelatinous states. Depending upon requirements, penicillin as an antibiotic was added to reach 0.2 weight %

TABLE 2

| Examples | Powder (g) | Water (ml) |
|---|---|---|
| Application | Preparation Example of | 90 |
| Example of | standard group, 10.0 | |
| standard group | | |
| Application | Preparation Example 1, | |
| Example 1-1 | 10.0 | |
| Application | Preparation Example, 20.0 | |
| Example 1-2 | | |
| Application | Preparation Example 2, | |
| Example 2 | 10.0 | |
| Application | Preparation Example 3, | |
| Example 3 | 10.0 | |
| Application | Preparation Example 4˜13, | |
| Example 4˜14 | 10.0 | |

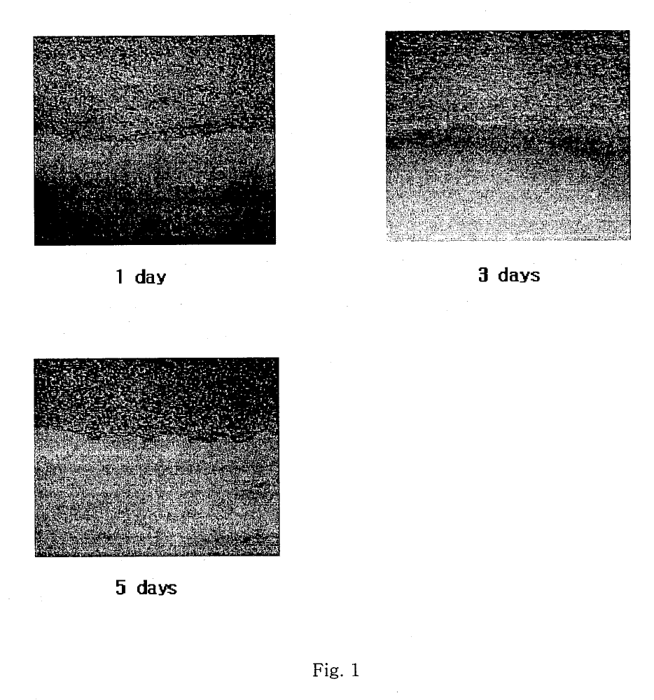

As experimental cells, smooth muscle cell and fibroblast cell were adopted. These cells were poured in 5×10⁵ cells and cultivated onto polystylene dish for cell culture with 10 cm of radius and then removed when cells grew to about 0.5 cm of width by using pincet. Afterward, the anti-adhesion barrier prepared in Preparation Example was administered. The treated cells were continued to be cultivated, observed intermittently under a microscope whether the cells can penetrate the covering layer and proliferate and taken to pictures (See FIG. 1). In FIG. 1, each date depicted days lapsed after the anti-adhesion barrier was administered and the upper region of the picture was a cell layer, the lower region of the picture, a covering layer.

As a result, the diffusion of animal cells were blocked in all Examples until 5 days lapsed after cultured. All the experimental results were very similar and thus only one set of the picture that showed the experimental result of Application Example 1-1 were attached in FIG. 1.

As illustrated in FIG. 1, at each step of the observation, a cell domain and a gel application domain were separated definitely with making a border line, which showed that cells could not penetrate a gel application domain.

Therefore, all the anti-adhesion barriers of the present invention were confirmed to have anti-adhesion effects during a short period.

### Toxicity Experiment Example

(1) The compositions manufactured in above mentioned Application Examples were investigated whether it could have the toxicity against animal cells. According to the prescriptions of ISO 10993-5, the toxicity experiment was accomplished. L929 cells were cultivated to form a monolayer by using MEM media, treated with trypsin, measured to have 1×10 cells/ml of cell concentration and then cultivated again to form a monolayer. Afterward, the anti-adhesion barrier, adcon, commercially available and manufactured in Gliatech company and the compositions prepared in Application Examples (See Table 3) were added in 4 ml respectively, cultivated in 5% CO₂ incubator at 37° C. and then cell concentrations were estimated in the culture broth after 24 hours and 48 hours. In Toxicity Experiment Example of standard group, the cell concentration at 48 hours lapse was decided to become 100 and the relative concentrations against this value were depicted in following Table 3.

TABLE 3

Relative
concentration of
cells

| | | After 24 | After 48 |
|---|---|---|---|
| Examples | Additives | hrs. | hrs. |
| Toxicity Example | Application Example | 95 | 100 |
| 1 of standard | of standard group | | |
| group | | | |
| Toxicity Example | adcon | 87 | 85 |
| 2 of standard | | | |
| group | | | |
| Toxicity Example | Application Example | 114 | 108 |
| 1 | 1-1 | | |
| Toxicity Example | Application Example | 119 | 121 |
| 4 | 4 | | |
| Toxicity Example | Application Example | 125 | 130 |
| 5 | 5 | | |
| Toxicity Example | Application Example | 92 | 95 |
| 6 | 6 | | |
| Toxicity Example | Application Example | 97 | 96 |
| 7 | 7 | | |
| Toxicity Example | Application Example | 101 | 105 |
| 8 | 8 | | |
| Toxicity Example | Application Example | 94 | 101 |
| 9 | 9 | | |
| Toxicity Example | Application Example | 96 | 115 |
| 10 | 10 | | |
| Toxicity Example | Application Example | 90 | 92 |
| 11 | 11 | | |
| Toxicity Example | Application Example | 99 | 101 |
| 12 | 12 | | |
| Toxicity Example | Application Example | 101 | 92 |
| 13 | 13 | | |
| Toxicity Example | Application Example | 103 | 108 |
| 14 | 14 | | |

As illustrated in Table 3, various compositions of the anti-adhesion barriers in the present invention did not depict toxicities high enough to inhibit the growth of animal cells. On the other hand, presently commercially available anti-adhesion barrier, adcon produced in Gliatech company was confirmed to have a considerable toxicity against animal cells.

(2) As a representative case from various compositions of the present invention, various levels of toxicities in compositions corresponding to Application Example 1-1 of Table 2 demonstrated above were analyzed accurately. The analysis was performed by investigating the stability/efficacy in KOREA TESTING AND RESEARCH INSTITUTE FOR CHEMICAL INDUSTRY.

Consequently, as illustrated in following Table, the compositions of the present invention were identified to indicate any abnormality in various toxicity experiments.

TABLE 4

| Items of tests | Test Method | Test Result |
|---|---|---|
| Cytotoxicity Test | Notification of | No |
| | Korea food & | abnormal result |
| | Drug | |
| | Administration | |
| | ISO 10993-5 | |
| Skin sensitization | ISO 10993:10 | No |
| test | | abnormal result |
| Acute toxicity test | Notification of | No |
| | Korea food & | abnormal result |
| | Drug | |
| | Administration | |
| Genetic toxicity test | USP NF (1998) | No |
| (carcinogenicity test) | | abnormal result |
| Transplantation test | USO NF (1998) | No |
| | | abnormal result |
| Subacute toxicity test | Notification of | No |
| | Korea food & | abnormal result |
| | Drug | |
| | Administration | |
| Chronic toxicity test | Notification of | No |
| | Korea food & | abnormal result |
| | Drug | |
| | Administration | |

In the meantime, substances supplemented additionally in Application Example 4˜14 (respectively, interleukine-10, hyaluronate, heparin, nifedipine HCl, phosphatidylcholine, medroxyprogestone acetate, L-arginine, nitric oxide, PVA, protein inhibitor and dextran) were known to be compatible to a human body without various levels of toxicity.

Hence, the various compositions of anti-adhesion barriers in the present invention did not cause side effects according to toxicity although administered in a body.

### Application Example

### In vivo Experiments

In the compositions prepared in Application Examples described above, the preventive effects upon anti-adhesion were investigated by being administered into animal bodies.

(1) The effects upon anti-adhesion were examined in gel type compositions of anti-adhesion barrier manufactured in Application Example 1-1, 1-2 and 2 demonstrated in Table 2. For convenience of experiments, penicillin as an antibiotic for microbes was added with 0.2 weight %.

Mice aged 8˜24 weeks (weight: 220˜450 g, male) were cut on a dorsal region and thus panniculus adiposus was made to be bleeded by affecting small trauma around blood vessels of muscles. Then, the gel type anti-adhesion barrier was applied and sealed to be recovered forward original position.

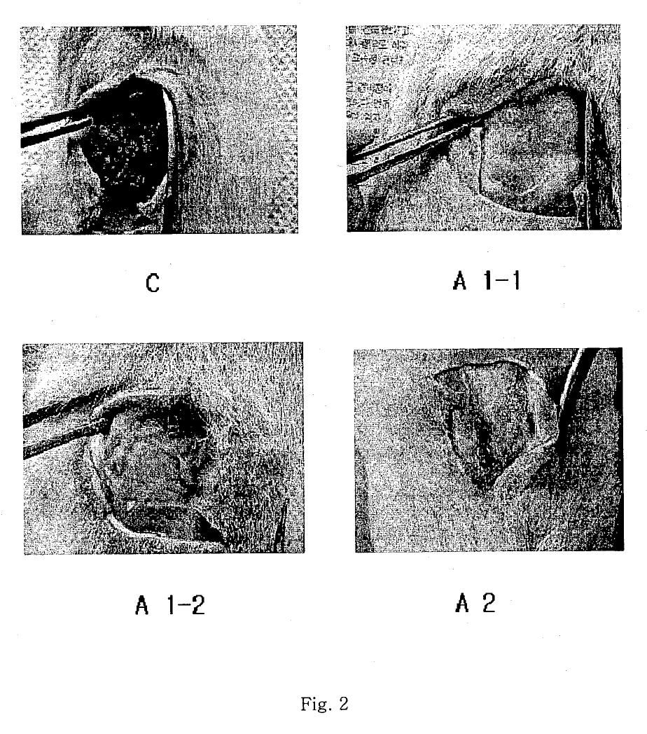

After one month from the operation, the above-mentioned site of the operation was opened with a mess and then investigated so that the anti-adhesion effect and haemostatic effect were shown in FIG. 2 as a picture. In this picture, symbol C, A, 1-1, A, 1-2 and A2 depicted states that gels of the anti-adhesion barrier prepared in Application Example of standard group, Application Examples 1-1, 1-2 and 2 were applied.

As pictures attached to FIGs, when the anti-adhesion barriers of the present invention comprising NaCMC and gellan gum were administered (A 1-1, A 1-2 and A 2), the adhesion caused at the sutured portion by the first operation was not found and symptoms such as bleeding or inflammation were never discovered after a sutural operation. On the contrary, when typical anti-adhesion barriers known already (comprising only NaCMC, C) were administered, the adhesion caused at the sutured portion by the first operation was found and symptoms such as bleeding or inflammation were discovered after a sutural operation.

(2) In the compositions as demonstrated in Table 5 as follows, the preventive effects upon anti-adhesion were investigated.

Mice aged 8 weeks (SD rat, 280˜400 g, male) were opened on a dorsal region with a mess and muscles around the spine were cut to about 0.5 cm of depth and 1 cm of length. Then, the gel type anti-adhesion barrier was applied and sealed to be recovered forward the original position.

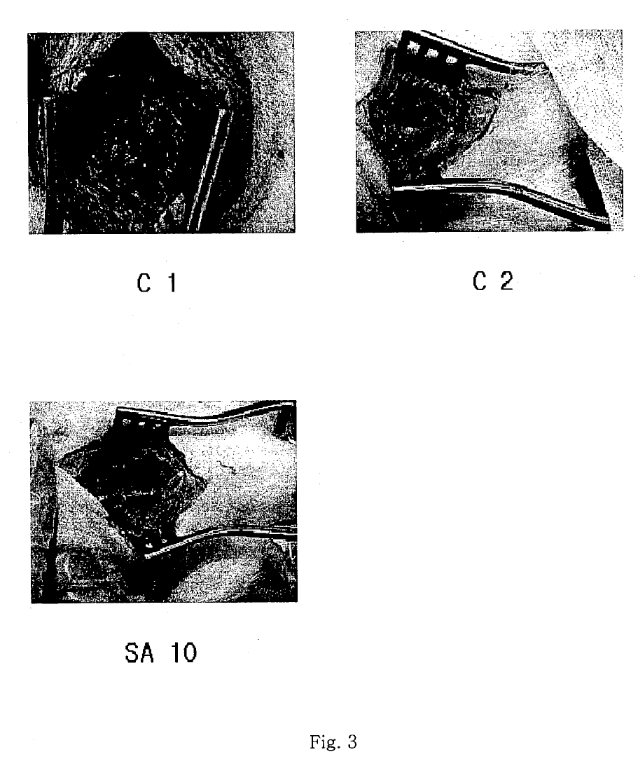

After 8 weeks from the operation, the above-mentioned site was opened with a mess and then investigated so that the severity of adhesion was measured and taken to a picture (See FIG. 3). In Application Example 1-1˜14 for spines, almost similar appearances were identified and only a representative picture of these was attached. In the picture, C 1, C 2 and SA 10 were observed respectively in samples corresponding to Application Example 2 of spine standard group and Application Example 10 of spines.

The degees of adhesion were estimated objectively by performing a double blind method. In detail, the observation by naked eye was performed and gray holtz grade which was decided through a tenacity at an adhesion site were measured. Estimation of five stages was accomplished so that the sample in which the adhesion effect was not found was defined as 0 and the sample in which an extension dense adhesion was found to be very strong was defined 4. In 10 samples, average gray holtz grade was demonstrated in Table 5.

TABLE 5

| | Components | | gray |
|---|---|---|---|
| | | Water | holtz |
| Examples | Powder (g) | (ml) | grade |
| Application Example | Preparation Example | 90.0 | 3.65 |
| 1* of spine standard | 1 of standard group | | |
| group | | | |
| Application Example 2 | Adcon (1) | — | 1.4 |
| of spine standard | | | |
| group | | | |
| Application Example | Preparation Example | 90.0 | 0.25 |
| 1-1* of spine | 1, 10.0 | | |
| Application Example | Preparation Example | | 0.2 |
| 1-2 of spine | 1, 20.0 | | |
| Application Example 2 | Preparation Example | | 0.4 |
| of spine | 2, 10.0 | | |
| Application Example 3 | Preparation Example | | 0.2 |
| of spine | 3, 10.0 | | |
| Application Example 4 | Preparation Example | | 0.1 |
| of spine | 4, 10.0 | | |
| Application Example 5 | Preparation Example | | 0.0 |
| of spine | 5, 10.0 | | |
| Application Example 6 | Preparation Example | | 0.2 |
| of spine | 6, 10.0 | | |
| Application Example 7 | Preparation Example | | 0.3 |
| of spine | 7, 10.0 | | |
| Application Example 8 | Preparation Example | | 0.4 |
| of spine | 8, 10.0 | | |
| Application Example 9 | Preparation Example | | 0.5 |
| of spine | 9, 10.0 | | |
| Application Example | Preparation Example | | 0.3 |
| 10 of spine | 10, 10.0 | | |
| Application Example | Preparation Example | | 0.2 |
| 11 of spine | 11, 10.0 | | |
| Application Example | Preparation Example | | 0.5 |
| 12 of spine | 12, 10.0 | | |
| Application Example | Preparation Example | | 0.0 |
| 13 of spine | 13, 10.0 | | |
| Application Example | Preparation Example | | 0.1 |
| 14 of spine | 14, 10.0 | | |

*20 samples were applied, in others 10 samples were applied.

(1) The anti-adhesion barrier, adcon which is manufactured and sold from Gliatech company was administered through prescription for use.

As illustrated Table 5, when the anti-adhesion barriers of the present invention comprising various compositions were applied, the adhesion between tissues was rarely observed. On the contrary, when conventional anti-adhesion barriers already known were administered (Application Example 1 of spine standard group 1 and Application Example 2 of spine standard group), adhesions were found in a considerable ratio. Especially, the conventional anti-adhesion barrier comprising only NaCMC (Application Example 1 of spine standard group) was shown to have a severe adhesion between tissues.

As pictures attached to FIGs, when the anti-adhesion barriers of the present invention comprising NaCMC and gellan gum were administered (SA 10), the adhesion caused at the sutured portion by the first operation was not found and symptoms such as bleeding or inflammation were never discovered after a sutural operation. On the contrary, when typical anti-adhesion barriers known already (comprising only NaCMC and adcon; C 1 and C 2 in FIG) were administered, the adhesion was caused at the sutured portion and symptoms such as bleeding or inflammation were found.

Consequently, when only NaCMC was contained, this polymer was absorbed into a body so fast that the anti-adhesion effect was identified not to be enough (Fertil. Steril., 1984, June, 41: 6, 926-928; Fertil. Steril., 1984, June, 41: 6, 929-932; Am. J. Obstet. Gynecol., 1986, 155: 3, 667-670). Besides, the anti-adhesion barrier of the present invention in which gellan gum was added in order to control the absorption of NaCMC into a body, sustain the shape and lubrication of gel was identified to compensate disadvantages of typical NaCMC completely.

Furthermore, the anti-adhesion barrier composition of the present invention was verified to have more outstanding effect than commercially available anti-adhesion barrier adcon already known.

(3) In addition, the preventive effects upon anti-adhesion were investigated in detail by using the compositions of Application Example 1 (namely, the anti-adhesion barrier comprising NaCMC) and one composition of the present invention.

Mice aged 8 weeks (SD rat, 280˜400 g, male) were opened on an abdominal region with a mess and blood vessels situated in between abdominal skin and organs were touched to have a small injury and bleeded. Then, the composition of Application Example 1 of standard group and the composition of Application Example 1-1 were applied and sealed to be recovered forward the original position. At each composition, the number of samples was adjusted to 3.

After 2, 4 and 8 weeks from the operation, the above-mentioned site was opened and then investigated whether adhesions were present or not (See FIG. 4). In FIG. 4, 1-1 denoted Application 1-1 and the standard group denoted Application Example 1 of standard group. As illustrated in attached pictures, when the anti-adhesion barriers of the present invention were applied, the adhesion in between abdominal skin and organ was not observed in spite of 8 week lapse. On the contrary, when the anti-adhesion barrier of Application Example 1 of standard group (namely, the anti-adhesion barrier comprising only NaCMC), the adhesion was identified to happen after 2 week lapse very strongly in between abdominal skin and organ.

Therefore, the gellan gum which was added in the composition of the present invention was verified once more to have preventive effects upon adhesion in between tissues since it should control the absorption of NaCMC into a body and lubrication of gel be improved.

(4) In addition, the preventive effects upon adhesion were investigated in detail by using conventional, commercially available anti-adhesion barrier adcon (product from Gliatech company) and one composition of the present invention (the composition in Application Example 1-1).

Mice aged 8 weeks (SD rat, 280˜400 g, male) were opened on an abdominal region with a mess and blood vessels of muscles around spine were affected to have a small injury and bleeded. Then, adcon was applied through prescription for use and the gel composition of Application Example 1-1 were applied and sealed to be recovered forward the original position. At each composition, the number of samples was adjusted to 2.

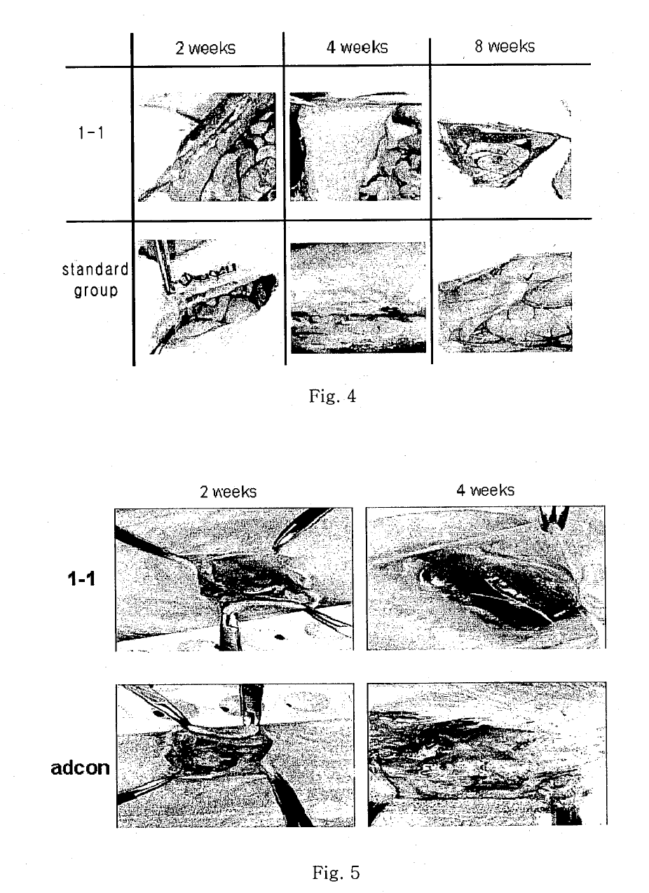

After 4 and 8 weeks from the operation, the above-mentioned site was opened again and then investigated the adhesion degree and the presence of bleeding (See FIG. 5). In FIG. 5, 1-1 denoted Application Example 1-1 and adcon denoted the case for applying adcon. As illustrated in attached pictures, when the anti-adhesion barriers of the present invention were applied, the adhesion in between abdominal skin and organ was not observed in spite of 4 week lapse and also bleeding did not happen during the process for separating tissues. But, in the sample that adcon was applied, the adhesion was identified to happen after 2 week lapse in between skin tissue and muscle tissue and the adhesion in between skin tissue and muscle tissue become stronger in a degree so that bleeding was caused when separated from tissues.

Therefore, the composition of anti-adhesion barrier in the present invention was verified to have more outstanding effects upon anti-adhesion than conventional, commercially available anti-adhesion barrier adcon.

<In vivo Degradation Experiment of Anti-Adhesion Barrier>

The anti-adhesion barrier of the present invention was investigated about the mechanism and the degradation according to time lapses when administered into a body.

15 Mice aged 8 weeks (SD rat, 280˜400 g, male) were cut on an abdominal region with a mess and opened in between abdominal skin and panniculus adiposus with about 5 cm of radius. Then, blood vessels of muscles around spine were affected to have a small injury and bleeded. Afterward, the composition of Application Example 1-1 of abdominal region described above (gel comprising NaCMC 5.0 g, gellan gum 5.0 g and 90 ml of water) was applied in 10 ml to a interval between opened abdominal skin and panniculus adiposus and sealed to be recovered forward the original position of skin.

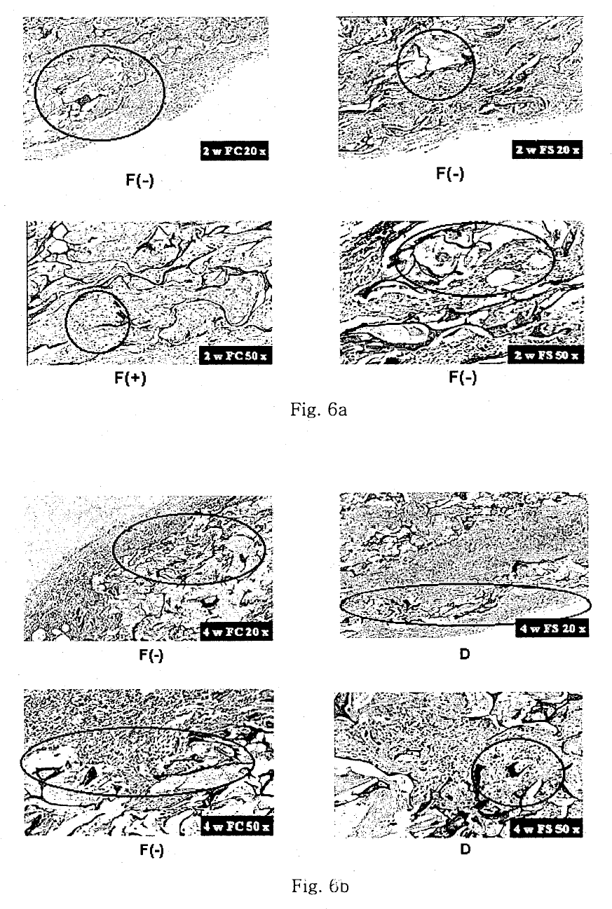

After 2, 4 and 8 weeks from the operation, the above-mentioned sites was collected from 5 mice respectively and then treated and stained according to typical sampling method of animal tissue. Afterward, the result was observed under a microscope in 80 and 200 X and taken to pictures (See 6a and 6b). FIG. 6a depicted the result of 2 week lapse after administered and FIG. 6b, the result of 4 week lapse after administered. In FIG. 6, F(+) was an appearance that fibrosis phenomenon was induced; F(−), an appearance that fibrosis phenomenon was inhibited; D, an appearance that the anti-adhesion barrier was degraded. In this picture, white color denoted an anti-adhesion barrier; red color, gel in which an anti-adhesion barrier and secreted blood were conjugated; black dot denoted a fibrosis and remained portion, a tissues of body.

As illustrated in FIGs, the adhesion between tissues of operated legions abdominal skin was observed to be prevented in spite of 2 and 4 week lapse from the operation. Then, after 4 weeks from the operation, the considerable part of anti-adhesion barrier was degraded and absorbed but the adhesion between tissues was identified not to be caused at all. Although illustrated above, after 6 weeks form the operation the anti-adhesion barrier was rarely discovered.

In detail, when the composition of the present invention was administered, tissues at a legion site affected through the operation were not adhered and on the contrary, was confirmed that it can be sustained for a period sufficient to be recovered to original state and degraded and absorbed.

### INDUSTRIAL APPLICABILITY

As illustrated and confirmed above, the anti-adhesion barriers of the present invention have an outstanding anti-adhesion effect and haemostatic effect since sodium carboxymethyl cellulose (NaCMC) which has a remarkable anti-inflammation effect, anti-bacterial property, regenerative effect upon a legion site, haemostatic effect and the like and also outstanding suitability for a human body and safety, is a major component. Besides, the anti-adhesion barriers of the present invention is a gel type and contains gellan gum enhancing lubrication (flowing) and antibiotics and thus has a high attachment, transparency, flowing, application, affinity and the like.

Those skilled in the art will appreciate that the conceptions and specific embodiments disclosed in the foregoing description may be readily utilized as a basis for modifying or designing other embodiments for carrying out the same purposes of the present invention.

Those skilled in the art will also appreciate that such equivalent embodiments do not depart from the spirit and scope of the invention as set forth in the appended claims.

What is claimed is:

1. An anti-adhesion barrier comprising sodium carboxymethyl cellulose (NaCMC) having a regenerative function in a legion site, haemostatic function, compatibility for a human body, and safety and gellan gum controlling absorption of said sodium carboxymethyl cellulose into a human body in a weight ratio of 1:(0.2˜5).

2. The anti-adhesion barrier according to claim 1, in which a composition of sodium carboxymethyl cellulose and gellan gum is in a weight ration of 1:(0.8˜1.25).

3. The anti-adhesion barrier according to claim 1 or claim 2, in which one or more than two substances selected from a group comprising interleukine-10, hyaluronate, heparin, nifedipine HCl, phosphatidylcholine, medroxyprogestone acetate, L-arginine, nitric oxide, polyvinylalcohol (PVA), protein inhibitor and dextran are contained additionally in a weight ratio of 1:(0.2˜5) against said sodium carboxymethyl cellulose.

4. The anti-adhesion barrier according to claim 1 or claim 2, in which antibiotics are contained additionally in a weight ratio of 1:(0.02˜0.06) against said sodium carboxymethyl cellulose.

5. The anti-adhesion barrier according to claim 1 or claim 2, in which said anti-adhesion barrier is a foam type, powder type or gel type.

6. The anti-adhesion barrier according to claim 5, in which said foam type of anti-adhesion barrier is prepared by a process, comprising steps as follows: (1) injecting a mixed solution consisting in components and adequate amount of water into a container having a suitable shape and size; (2) freezing rapidly by submersing into liquid nitrogen; and (3) lyophilizing in a reduced pressure at −55° C. for more than 48 hours.

7. The anti-adhesion barrier according to claim 6, in which one step autoclaving at 120˜150° C. for 5˜20 minutes before freezing said mixed solution rapidly is added.

8. The anti-adhesion barrier according to claim 5, in which said powder type of anti-adhesion barrier is prepared by pulverizing said powder type of anti-adhesion barrier of claim 6 to reach a radius of particles in 0.2˜0.6 mm.

9. The anti-adhesion barrier according to claim 5, in which said powder type of anti-adhesion barrier is prepared by pulverizing said powder type of anti-adhesion barrier of claim 7 to reach a radius of particles in 0.2˜0.6 mm.

10. The anti-adhesion barrier according to claim 5, in which said gel type of anti-adhesion barrier is prepared by a process comprising steps as follows: (1) adding said powder type of anti-adhesion barrier of claim 8 in 1 weight portion against 4˜19 weight portion of distilled water and (2) stirring.

11. The anti-adhesion barrier according to claim 5, in which said gel type of anti-adhesion barrier is prepared by a process comprising steps as follows: (1) adding said powder type of anti-adhesion barrier of claim 9 in 1 weight portion against 4˜19 weight portion of distilled water and (2) stirring.
